(19) United States
(12) Patent Application Publication
(10) Pub. No.: US 2023/0218218 A1
(43) Pub. Date: Jul. 13, 2023

(54) MAGNETOENCEPHALOGRAPHY APPARATUS AND METHOD

(71) Applicant: Megin Oy, Helsinki (FI)

(72) Inventors: Jukka Nenonen, Helsinki (FI); Matti Kajola, Helsinki (FI); Samu Taulu, Seattle, WA (US)

(21) Appl. No.: 18/001,553

(22) PCT Filed: Jun. 23, 2021
(86) PCT No.: PCT/FI2021/050479

§ 371 Date: Dec. 12, 2022

(30) Foreign Application Priority Data
Jun. 25, 2020 (FI) 20205675

(51) Int. Cl. A61B 5/245; A61B 5/00

(52) U.S. Cl. CPC A61B 5/245; A61B 5/4064; A61B 5/7203; A61B 2562/0223; A61B 2562/046

(57) ABSTRACT

Disclosed is a magnetoencephalography apparatus (100) and a method. The apparatus comprises a plurality of magnetic sensors, one or more processors and one or more memories. The method comprises obtaining a reference data, calculating from the reference data a reference basis, obtaining a source basis, obtain a source data, adding together the source basis and the reference basis to form a joint basis and determine an estimate for the magnetic brain activity of the source by parametrizing the source data in the joint basis.

### FIELD

The present invention relates to magnetic brain imaging.

### BACKGROUND

With current technology, magnetic brain imaging is performed using very sensitive measurements requiring a large set of measurement sensors, which provide a number of measurement channels sufficient to achieve the required sensitivity. Sources of noise and measurement artifacts are various and can emerge from the person to be measured, from the measurement environment or from the measurement equipment itself. This is due to the magnetic field of a human brain being very small in itself and considerably smaller than the ambient magnetic noise in an urban environment.

Various means are used to reduce noise during measurement, each of the means having its own shielding factor. As a first example, cryogenic cooling of measurement sensors is often used, particularly for SQUID sensors. As a second example, optically pumped magnetometer (OPM) measurement sensors are used and placed closer to the scalp than SQUID sensors. As another example, passive shielding means include performing the measurement in a magnetically shielded room (MSR). Active shielding means, including external active shielding (EAS) and internal active shielding (IAS), can be performed with a set of coils compensating for external interference at the measurement site, which can be used to allow the operation of the measurement sensors at their dynamic range. An additional example is a reference sensor assembly, which can be used at the vicinity of the actual measurement sensors allowing the assembly to be configured for detecting only the remaining external interferences, which can thereafter be subtracted from the signal of the measurement sensors to yield a more accurate measurement result. Finally, the noise can be reduced by signal processing for the measurement sensors, where techniques such as Signal Space Separation (SSS), Signal Space Projection (SSP) and independent component analysis (ICA) have been used. The aforementioned means of noise reduction are generally used together to complement each other.

Currently, signal processing in state of the art systems may be based on the SSS method, which has been disclosed, for example, in WO2004081595A1. The benefit of the method is not only that it can provide a relatively large shielding factor but also that it can be evolved and combined with various additional developments to more closely adapt the method to the non-idealities of the actual measurement environment. However, a handicap is that to get full advantage of a noise reduction method that is based on deterministic SSS modelling, a fine calibration is required. Since the fine calibration requires a qualified technician, it is typically performed only upon the installation of the system at the point of use with possible recalibration only during intermittent maintenance of the system, for example once a year.

With the development of the noise-reduction methods that are based on SSS, a shielding factor of 30-40 can currently be reached for the SSS-based method with factory calibration in a magnetoencephalography (MEG) system with 306 channels. With fine calibration, the shielding factor for an SSS-based method can exceed 100.

### OBJECTIVE

An objective is to alleviate the disadvantages mentioned above.

In particular, it is an objective to provide an apparatus and a method for magnetoencephalography, which do not involve the fine calibration of an SSS-based method.

Additionally, it is an objective to provide an apparatus and a method for magnetoencephalography, which can be used to simultaneously scan the entire brain with less than 306 channels.

Finally, it is an objective to provide a novel apparatus and method for noise-reduction in magnetoencephalography, particularly in view of the traditional SSS method or methods based on the SSS method.

### SUMMARY

An MEG recording is a measurement performed by an MEG apparatus, which recording may be used to determine magnetic brain activity. In the recording, an interference contribution is always present even if the contribution has been largely suppressed by one or more noise reduction means such as MSR, IAS or EAS. In the presence of magnetic brain activity, the recording comprises both the interference contribution and a contribution from the magnetic brain activity.

When an SSS-based method, including the original SSS method, is used the signal processing actually divides the interference contribution into external interference and internal interference, where external interference includes all magnetic signals emanating from the surroundings of the measurement equipment, such as magnetic pollution due to power lines, radio communication, traffic, elevators and so forth. In short, SSS-based methods involve dividing space into three different regions with a first region corresponding to the space for the measurement subject, a second region corresponding to the space for the measurement equipment, which is positioned around the measurement subject and a third region corresponding to the space outside the measurement equipment. The fine calibration process of an SSS-based method then involves optimizing several coefficients to, for example by individually rotating the normal unit vectors of each measurement sensor in turn in small steps to find the best match between measured and modelled sensor data.

In contrast, the present disclosure allows the deterministic SSS method, together with its requirement of fine-calibration, to be disposed of. According to a first aspect, a magnetoencephalography apparatus (“the apparatus”) comprises a plurality of magnetic sensors (“the sensors”) arranged for measurement of magnetic brain activity originating within a first volume. The plurality of magnetic sensors is arranged for positioning within a second volume, which is outside the first volume. This allows the plurality of magnetic sensors to substantially surround the first volume. The apparatus comprises one or more processors coupled to the plurality of magnetic sensors for controlling the measurement of magnetic brain activity and one or more memories comprising computer program code. The one or more memories and the computer program code are configured to cause the one or more processors to perform the following in the indicated order or in any other suitable order. Any or all of the following may also be performed independent from the apparatus as a method of its own.

First, obtain a reference data corresponding to one or more measurements of the plurality of magnetic sensors in the absence of sources of magnetic brain activity in the first volume (“reference measurement”). This allows forming a MEG recording of the actual measurement environment for the apparatus since the reference data comprises an interference contribution both from the interference external to the apparatus and from the interference originating from the apparatus itself.

Second, calculate from the reference data a first basis (“reference basis”), which represents magnetic activity in the absence of sources of magnetic brain activity in the first volume, in a signal space defined by the plurality of magnetic sensors. This allows the interference contribution to be divided into groups, each group corresponding or at least substantially corresponding to one or more interference sources, for example a power line or traffic. While the magnitude of an interference signal corresponding to an interference source may change over time, an interference signal corresponding to an interference source has a characteristic magnitude distribution across the plurality of magnetic sensors, which is typically different for different interference sources. This allows the magnitude distribution to be identified as corresponding to a particular interference source.

Third, obtain a second basis (“source basis”), which represents magnetic brain activity of a human brain positioned in the first volume, in the signal space defined by the plurality of magnetic sensors. The source basis can be formed utilizing the knowledge of a human brain and laws of physics, i.e. it can be formed without any measurements of the present source. It is a calculated basis and typically can be solely based on numerical analysis but it could also be based, partially or fully, on measurements of one or more reference subjects. The source basis allows determining the contribution from magnetic brain activity using information of the characteristic magnetic field distributions generated by a human brain. For example, a signal corresponding to magnetic brain activity in one part of a human brain has a characteristic magnitude distribution across the plurality of magnetic sensors, which is typically significantly different for different parts of a human brain and from the characteristic magnitude distribution of any interference signals across the plurality of magnetic sensors.

Fourth, obtain a source data corresponding to one or more measurements of the plurality of magnetic sensors in the presence of a source of magnetic brain activity in the first volume (“source measurement”). This allows forming a MEG recording where a contribution from magnetic brain activity is present, together with an interference contribution corresponding to the time of the source measurement.

Fifth, add together the source basis and the reference basis to form a joint basis in the signal space defined by the plurality of magnetic sensors. This combination allows the source basis to solely correspond to the contribution from the magnetic brain activity so that no fine calibration corresponding to that of the SSS-based methods is needed. Correspondingly, the reference basis can solely correspond to the interference contribution. The joint basis can then be composed as a direct combination of the source basis and the reference basis so that the basis vectors of the joint basis comprise the basis vectors of the source basis and basis vectors of the reference basis. The number of basis vectors for the joint basis can then be the sum of the number of basis vectors for the source basis and the reference basis. It is emphasized that adding together the bases does not imply the mathematical addition of individual basis vectors but it may herein refer to a combination of bases to form a joint basis comprising basis vectors from both the source basis and the reference basis. The joint basis may therefore have a dimension larger than the dimension of the source basis and the dimension of the reference basis. An orthogonalization for the joint basis may be performed, for example when determining a pseudo-inverse for the joint basis. Such an othogonalization may define a set of non-zero eigenvalues, which may be considered as an effective dimension of the joint basis. The effective dimension of the joint basis may be equal to or smaller than the sum of the dimensions of the source basis and the reference basis. The joint basis may span or substantially span the signal space defined by the plurality of magnetic sensors. However, the number of basis vectors of the joint basis may also be smaller or even substantially smaller than the number of signal channels of the plurality of magnetic sensors. The joint basis is constructed, by the combination of the reference basis and the source basis, to allow the interference contribution to be separated from the contribution from the magnetic brain activity without generating a computational estimate for the interference contribution, particularly where the estimate requires determination of the exact position and/or orientation of the sensors, i.e. without a computational estimate requiring fine-calibration.

Sixth, determine an estimate for the magnetic brain activity of the source by parametrizing the source data in the joint basis. This allows estimating the interference contribution as the part of the source data which, by the parametrization, falls into the sub-basis corresponding to the reference basis. Correspondingly, the magnetic brain activity of the source can be estimated as a part of the source data which, by the parametrization, falls into the sub-basis corresponding to the source basis. Generally, the linear combination of these two parts still yields the original source data.

As stated above, the order of the steps may vary. As an example, the third step may be performed any time prior to forming the joint basis, for example before the reference measurement and/or after the source measurement. One or more source bases may even be pre-configured in the one or more memories. Correspondingly, the reference basis may be pre-configured in the one or more memories. Pre-configuration may have been performed on-site at the location where the apparatus is to be used or before the apparatus has been installed at the location where it is to be used.

According to a second aspect, a method comprises obtaining a reference data corresponding to one or more measurements of a plurality of magnetic sensors in the absence of sources of magnetic brain activity in a first volume, i.e. the reference measurement. In the reference measurement, the plurality of magnetic sensors have been arranged for measurement of magnetic brain activity originating within the first volume and positioned within a second volume, which is outside the first volume. The method also comprises calculating from the reference data a reference basis, which represents magnetic activity in the absence of sources of magnetic brain activity in the first volume, in a signal space defined by the plurality of magnetic sensors. The method comprises obtaining a source basis, which represents magnetic brain activity of a human brain positioned in the first volume, in the signal space defined by the plurality of magnetic sensors. The method also comprises adding together the source basis and the reference basis to form a joint basis in the signal space defined by the plurality of magnetic sensors. The method comprises obtaining a source data corresponding to one or more measurements of the plurality of magnetic sensors in the presence of a source of magnetic brain activity in the first volume, i.e. the source measurement. Finally, the method comprises determining an estimate for the magnetic brain activity of the source by parametrizing the source data in the joint basis.

What is described above in connection with the first aspect applies to the second aspect. In particular, this holds for the part that in the first aspect is performed by the one or more processors. Correspondingly, the steps of the method may be in the indicated order or in any other suitable order. Both the method and the apparatus can be adapted for magnetic brain imaging, specifically that of a human brain. Both involve one or more measurements of a plurality of magnetic sensors for a MEG recording in a first volume, which sensors define a number of signal channels (herein also “channels”) for the MEG recording. They may be adapted for simultaneous or substantially simultaneous imaging of an entire brain with less than 306 signal channels. An important effect that, for magnetic brain imaging, such as MEG recording, in accordance with the present disclosure, a requirement that the number of signal channels needs to be much larger than the sum of the dimensions of the reference basis and the source basis may be lifted. As an example, the number of signal channels may be less than twice the dimension of the joint basis and/or less than twice the sum of dimensions of the reference basis and the source basis. Nevertheless, the number of signal channels may still be equal or larger than the dimension of the joint basis or the sum of dimensions of the reference basis and the source basis. The method and the apparatus allow notable improvements to accuracy for estimating the magnetic brain activity of the source without utilizing a deterministic SSS-based method. In contrast to the various SSS-based methods currently used, they can be used without generating a computational estimate for the interference contribution. Because of this, there is no requirement for fine-calibration, like in said SSS-based methods, that would require determining the positions and/or orientations of the sensors for calibrating the numerical determination of external interference.

Any of the embodiments described herein are applicable to any of the aspects.

In an embodiment, the magnetic sensors of the plurality of magnetic sensors are either all magnetometers or all gradiometers. These may be dedicated sensors for a particular type of MEG recording. It has been found that in contrast to previous MEG apparatuses requiring more complicated multi-sensor arrangements for a MEG recording, the method comprising the six steps indicated to be performed by the one or more processors according to the first aspect allows utilizing such a uniform sensor configuration with surprising accuracy. The reference measurement and/or the source measurement can thereby be performed solely by magnetometers or solely by gradiometers.

In an embodiment, the magnetic sensors of the plurality of magnetic sensors are gradiometers. Using an all-gradiometer assembly as the plurality of magnetic sensors has been found to provide a surprisingly competent performance. Moreover, it allows reducing the requirements for magnetic shielding, for example in comparison when magnetometers are used as the magnetic sensors. In overall, the use of gradiometers has been found to improve the robustness of the measurements while simplifying the apparatus and reducing costs.

In an embodiment, the magnetic sensors of the plurality of magnetic sensors are planar gradiometers. This has been found to allow reducing the sensitivity of the gradiometers to low-order gradients, thereby making it possible to provide improved accuracy with a given number of basis vectors of the reference basis. Correspondingly, it may allow using a smaller number of basis vectors of the reference basis to reach a given level of performance or accuracy. In turn, this allows stabilizing the numerical determination of the estimate for the magnetic brain activity. As an alternative, some or all of the gradiometers may be axial gradiometers.

In an embodiment, the plurality of magnetic sensors is arranged to measure the magnetic brain activity with 48-256 signal channels. This allows significant reduction in the currently used systems with 306 channels. With typical measurement distances and sensor noise levels in current MEG devices, in particular ones being based on SQUID-sensors, it has been found that having 100 or more channels may still be used to provide an improvement in the performance of the system. Having 150 or more channels may be used to provide improvement in numerical stability. Nevertheless, the number may still be smaller than 220-256, for example.

In an embodiment, the apparatus is arranged to automatically perform the one or more measurements of the plurality of magnetic sensors to obtain the reference data. This allows the apparatus to automatically update the reference basis so that it may gather more information of the magnetic environment of the apparatus and/or adjust to changes in the magnetic environment.

The source basis may be determined in more than one manner to efficiently utilize knowledge of a human brain to estimate what kind of a signal is generated at the sensors. In any case, the source basis is determined for a source positioned in the first volume, enclosed by the volume for the sensors. In an embodiment, the source basis is obtained based on a deterministic solution to the Maxwell's equations for the magnetic brain activity of a human brain, where the equations may be solved under the static approximation. The solution can be, for example, a direct solution to the scalar Laplace equation for potential. The solution may be expressed as a series development, for example as an orthogonal function development and/or a Taylor series development. The solution may also be expressed as a harmonic function development, for example as a spherical harmonic function development. Correspondingly, the basis vectors of the source basis may be the basis vectors of vector spherical harmonic functions. In an embodiment, the source basis is obtained based on a source model for the magnetic brain activity of a human brain comprising one or more stochastically positioned sources of magnetic brain activity. The source basis may be obtained based on a calculation of lead-fields for the stochastically positioned sources. For example, the stochastically positioned sources may comprise current and/or magnetic dipoles. They may be positioned for example at the cortex and/or spread in the volume of the brain.

According to a third aspect, computer program product comprises instructions which, when the computer program product is executed by a computer, cause the computer to carry out the method according to the second aspect and/or any of its embodiments alone or in combination.

It is to be understood that the aspects and embodiments described above may be used in any combination with each other. Several of the aspects and embodiments may be combined together to form a further embodiment of the invention.

### BRIEF DESCRIPTION OF THE DRAWINGS

The accompanying drawings, which are included to provide a further understanding and constitute a part of this specification, illustrate examples and together with the description help to explain the principles of the disclosure. In the drawings:

FIG. 1 illustrates an apparatus according to an example in a side view,

FIG. 2 illustrates a method according to an example, and

FIG. 3 schematically illustrates an apparatus according to an example.

Like references are used to designate equivalent or at least functionally equivalent parts in the accompanying drawings.

### DETAILED DESCRIPTION

The detailed description provided below in connection with the appended drawings is intended as a description of examples and is not intended to represent the only forms in which the example may be constructed or utilized. However, the same or equivalent functions and structures may be accomplished by different examples.

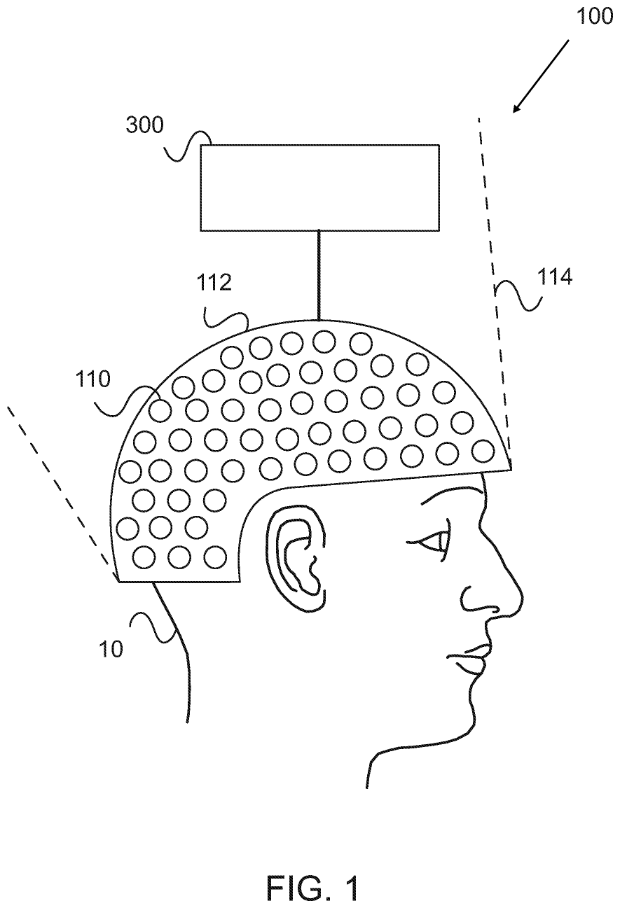

FIG. 1 shows an example of an apparatus 100, which can be a magnetoencephalography (MEG) apparatus. A measurement subject can be the brain of a human test subject 10. The apparatus 100 is arranged for measurement of the magnetic activity of a brain. For this purpose, the apparatus comprises a plurality of magnetic sensors 110 (“the sensors”), which are arranged for positioning proximate to the brain for measurement of brain activity. Since MEG is a noninvasive method, some or all of the sensors 110 may be positioned around the head of the test subject, for example so that the arrangement of the sensors 110 is substantially helmet-shaped. Some or all of the sensors 110 may also be positioned directly against the head of the test subject, for example when the corresponding sensors 110 are OPM sensors. This allows the positioning of the sensors 110 to adapt to the shape and/or size of the head. In an embodiment, the plurality of magnetic sensors 110 consist of or comprise OPM sensors 110. The apparatus 100 and/or the sensors 110 may be adapted for simultaneous or substantially simultaneous imaging of an entire brain.

A first volume is thereby a volume, where the brain is to be positioned and it may comprise an origin, for example substantially corresponding to the center point of the brain. The first volume is defined with respect to a second volume, where the sensors 110 are positioned during measurement. The first volume is thereby inside the second volume so that the second volume may enclose the first volume. The first volume may be substantially spherical. In this case, the origin may be located substantially at the center of the first volume. The union of the first volume and the second volume may be substantially spherical, in which case the origin may be located substantially at the center of the union. The first volume may be substantially the size of a human head. The second volume may be substantially the size of the volume required to contain the sensors 110, for example the size of a helmet or a MEG helmet positioned on a human head. The sensors 110 may be arranged to be positioned circumferentially or substantially circumferentially in the second volume. The sensors 110 may be arranged at one or more supports 112, for example a helmet-shaped support. This can be used to allow the positioning of the sensors 110 to substantially follow the curvature of a human head during measurement. The apparatus 100 may comprise a MEG helmet 114 comprising the support 112. The distances of the sensors 110 from each other and/or the origin are arranged to allow a MEG recording to be performed with the apparatus.

The sensors 110 may be magnetometers and/or gradiometers, in particular planar gradiometers. The apparatus 100 may be arranged to allow the measurement for brain activity to be performed using solely gradiometers or solely magnetometers. Each of the sensors 110 is arranged to provide one or more signal channels for measurement of magnetic brain activity and while the number of the sensors 100 may correspond to the number of signal channels, it is also possible to use multi-channel sensors providing more than one signal channel. However, the measurement of magnetic brain activity may be performed with a number of signal channels that is smaller than the previously used 306 channels. The number of signal channels may be less than 256, for a MEG recording of an entire brain. For example, the number of signal channels may be 48, 96, 148 or 220. With current levels of sensor noise, it has been found that using at least 96 signal channels provides a marked improvement in performance and using at least 148 signal channels may, in some embodiments, significantly improve the numerical stability of the signal processing. Naturally, the number of signal channels may be further increased to improve the capabilities of the apparatus 100. The number may be also larger than 306, or even larger than 700, for example for an apparatus 100 utilizing OPM sensors. The sensors 110 define a signal space as a space of magnetic signals measurable by the sensors 110. The signal space is a vector space and it can be spanned by a set of basis vectors. The number of basis vectors spanning the signal space may correspond to the number of signal channels. In practice, the effective dimension of the signal space useful for determining estimate for the magnetic brain activity may be smaller, even half of that or less.

The apparatus 100 may comprise a measurement device 300 arranged to collect measurement data from the sensors 110. While the measurement device 300 can be arranged connected to the sensors 110 with a wired and/or a wireless connection, using a wired connection allows reducing magnetic noise in the measurement environment.

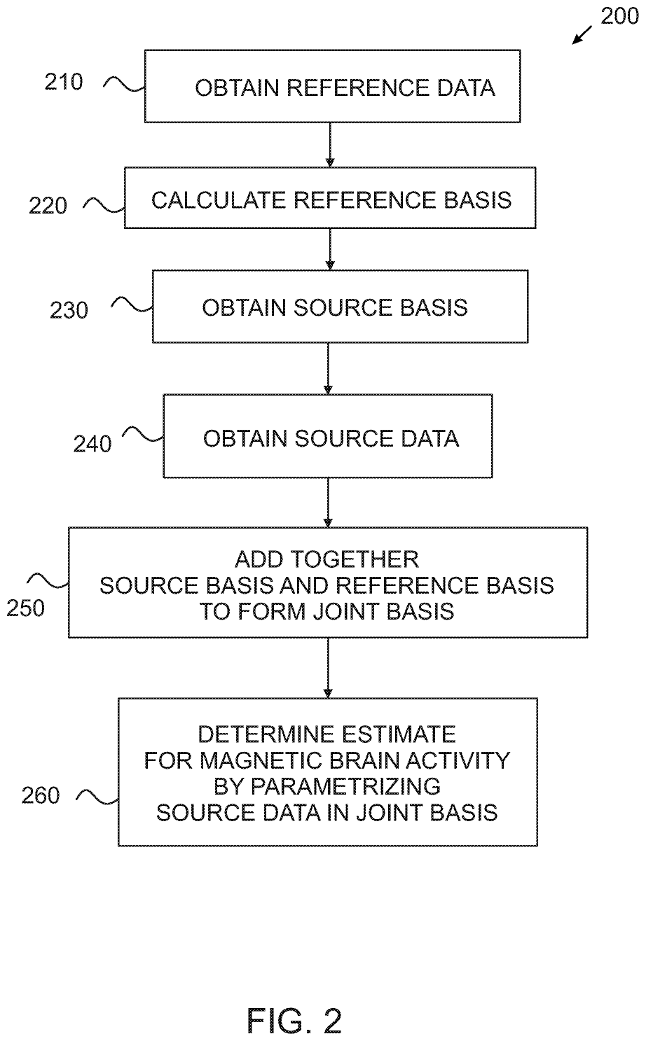

FIG. 2 shows an example of a method 200 for determining magnetic brain activity, or an estimate thereof, which can be adapted as a signal processing method. The magnetic brain activity is determined for a source, such as a human brain, positioned in a first volume, as described above for the apparatus 100, which may be used for performing any or all parts of the method 200. For measurement, a plurality of magnetic sensors 110 is used and the sensors 110 can be as described above. In particular, the sensors 110 are arranged to be positioned within the second volume as described above, so that they can be used for a MEG recording of a source in the first volume. The method comprises several parts which may be performed independently from each other and/or in any order.

In the method, reference data is obtained corresponding to a reference measurement 210 with the plurality of magnetic sensors 110. This reference data can be used to determine the magnetic environment of the first volume so that it can be taken into account when determining the magnetic brain activity of the source. Typically, the magnetic environment involves an interference contribution that may be several magnitudes larger than the contribution from the magnetic brain activity of the source. In addition, the reference data allows capturing any non-idealities in the apparatus 100 and/or the sensors 110 used to perform the measurements, in particular a source measurement, where a source of magnetic brain activity is present in the first volume. A reference measurement can be performed any time, for example before and/or after the source measurement. A reference measurement may comprise, for example an MEG recording of one or more minutes in the absence of sources of magnetic activity in the first volume. When the reference measurement is performed in an MSR, the MSR may be empty of sources of magnetic brain activity.

The reference data is used to calculate a reference basis 220, which represents magnetic activity in the absence of sources of magnetic brain activity in the first volume. This allows utilizing the whole reference measurement in construction of the reference basis. This way, the reference data, or the signals measured during the reference measurement, can be divided into a set of basis vectors and, optionally, normalized. The reference basis may be orthogonal. The reference basis may be formed, for example, using principal component analysis for the reference data. A covariance matrix can be computed from the reference data and principal component analysis (PCA) can be applied to determine the spatial patterns which characterize the reference data. The number of basis vectors of the reference basis n_{ref} may be the number of signal channels N minus the number of basis vectors of a source basis nₛ, i.e. n_{ref}=N−nₛ, but it can also be smaller since this only means that the signals measured during the reference measurement are divided in another manner. For example, one or more of the basis vectors may correspond to clear interference shapes corresponding to a specific source of interference whereas one or more may correspond to general background interference, where reducing the size of the reference basis may increase the part of the reference data allocated for the latter basis vectors. It has been found that it can, in some instances, be enough to use a limited number of basis vectors in the reference basis. For example, the number of basis vectors in the reference basis may be at least 5-8. It has been found that in several currently relevant embodiments, it suffices to use at most 15-50 basis vectors for the reference basis. The basis vectors of the reference basis correspond to the interference contribution, which may comprise all signals arising in the absence of a source of magnetic brain activity.

The method also comprises obtaining a source basis 330, which corresponds to the magnetic brain activity of a general human brain. One or more techniques for describing the magnetic activity produced by a human brain may be used. In particular, the source basis may be determined purely deterministically or it may be determined using a stochastic soured model for a human brain. The source basis may be orthogonal. The source basis may be constructed using, for example, a minimum of 20-30 basis vectors. This may allow a MEG recoding to be provided corresponding to an entire brain. For efficiency, the source basis may be constructed using a maximum of 100-120 basis vectors, for example. The source basis may be determined with respect to the origin, for example using a series development with respect to the origin. The source basis can be determined or re-determined at any point when the method is performed. The basis vectors of the source basis may correspond to magnetic fields, which are irrotational and sourceless outside the second volume. In an embodiment, the source basis is obtained based on a deterministic solution to the Maxwell's equations for the magnetic brain activity of a human brain. One example for a possible way of determining the basis vectors is given in “The magnetostatic multipole expansion in biomagnetism: applications and implications” by Jussi Nurminen, ISBN 978-952-60-5710-1 (section 3.2, which is hereby incorporated by reference). In an embodiment, the source basis is obtamed based on a source model for the magnetic brain activity of a human brain comprising one or more stochastically positioned sources of magnetic brain activity. One example for a possible way of determining the basis vectors is given in “The magnetostatic multipole expansion in biomagnetism: applications and implications” by Jussi Nurminen, ISBN 978-952-60-5710-1 (section 5.7, which is hereby incorporated by reference). In one more example, the source basis may be determined by using a source model where a layer of magnetic dipoles is positioned between regions corresponding to the white matter and the gray matter of the human brain. For the source models mentioned above, a lead-field matrix may be calculated for which eigenvectors can be determined, for example by using a singular-value decomposition, to determine the source basis.

In the method, source data is obtained corresponding to a source measurement 240 with the plurality of magnetic sensors 110. This source data can be used to determine the magnetic activity of the source, e.g. a human brain. A source measurement can be performed any time, for example before and/or after the reference measurement.

To optimize the accuracy of the description of the magnetic environment, the positioning and/or orientation of the sensors 110 can be substantially the same during the reference measurement 210 and the source measurement 240. The reference measurement and/or the source measurement can be performed simultaneously or at least substantially simultaneously for all signal channels. Both the reference measurement and the source measurement can be performed as an attempt to determine magnetic brain activity in the first volume allowing the two measurements to correspond to a substantially similar interference contribution. The source basis and/or the reference basis may be linearly independent. The joint basis may also be linearly independent.

The source data basis and the reference basis are added together to form a joint basis 250 in the signal space defined by the sensors 110. The joint basis thereby comprises the basis vectors of both the source basis and the reference basis but since they are separate, or linearly-independent in particular, any signal can be expressed in the joint basis separately as a contribution corresponding to the reference basis and a contribution corresponding to the source basis. Since the source measurement involves an interference contribution and a contribution from the magnetic brain activity of the source, the former can now described as the contribution corresponding to the reference basis, whereas the latter can now be described as the contribution corresponding to the source basis. The interference contribution is typically much larger than the contribution from magnetic brain activity so that the more accurately it can be estimated the more accurately the brain magnetic activity of the source can be determined. An estimate is determined by parametrizing 260 the source data in the joint basis. The estimate can be determined as the part of the source data which, when expressed in the joint basis, corresponds to the basis vectors of the source basis.

Overall, a magnetic signal can be expressed as a linear combination of a set of basis vectors each weighed by an amplitude coefficient. Therefore, the contribution from the brain magnetic activity of the source can be expressed as a linear combination of the source basis weighed by the amplitude coefficients that are obtained by parameterization of the source data in the joint basis. Correspondingly, a total magnetic field can be expressed as a linear combination of the basis vectors of the joint basis each weighed by their own amplitude coefficient. With multiple signal channels, this can be expressed as a matrix equation, where the magnetic field can be obtained as a product of a matrix corresponding to the joint basis and a vector corresponding to the amplitude coefficients. Correspondingly, solving the amplitude coefficients corresponds to inverting the matrix so that, typically, the parametrization involves inverting the matrix describing the joint basis. For determining the magnetic brain activity of the source it will naturally be enough to determine corresponding amplitude coefficients. The magnetic signal can be determined or extrapolated at any location in space, for example at the origin and/or at the location of a magnetic sensor. Non-idealities of the sensor array, such as uncertainty of exact sensor position, orientation and calibration, are embedded in the measured reference data and fine-calibration adjustments are therefore not needed. In fact, a gradiometric array could not even utilize the fine-calibration procedure currently used for SSS-based systems.

While not utilizing the SSS as such, the method can be used with all the major improvements available to the SSS method. In particular, the method allows compensating for signal disturbances caused by head movements inside the second volume. Moreover, disturbance signals from nearby interference sources, such as magnetized objects in subject's mouth or on the scalp, can be identified and the information can be used to improve the estimate for the magnetic brain activity. For this, methods similar to SSS expansions and time-domain subspace methods can be used. Temporal waveforms identified as disturbances can be projected out and interference-free MEG signals can be reconstructed using the source basis. In addition, the method can be extended with spatial means by augmenting the reference basis by adding one or more vectors, such as unit vectors, for isolating individual channels or one or more vectors identified in any way including a separate measurement and representing known disturbance which can be separately explained. Another spatial extension employs cross-validation for separating the uncorrelated channel-specific noise signals. The method can also utilize covariance-based a priori information in defining the amplitude coefficients of the source basis for reducing the background noise of the signals.

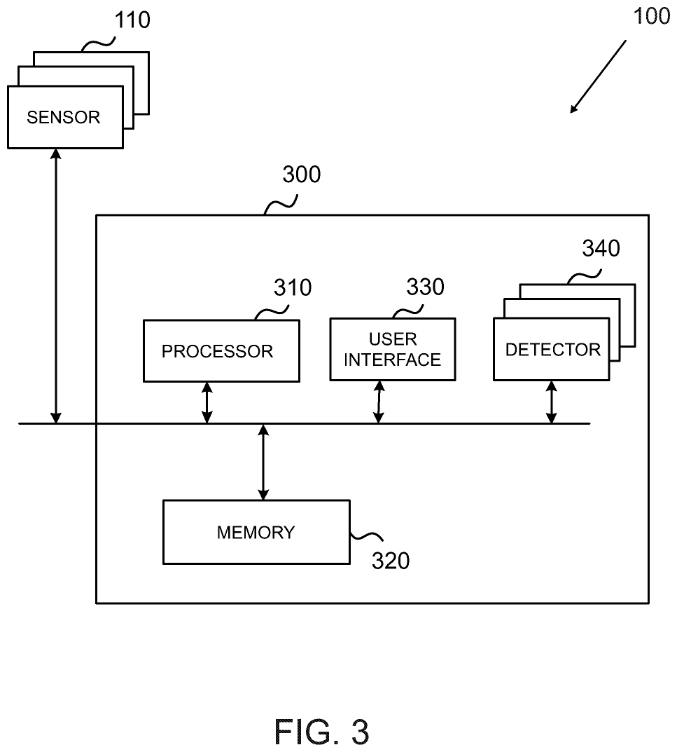

FIG. 3 shows an example of an apparatus 100. The apparatus 100 comprises one or more processors 310 and one or more memories 320 comprising computer program code. These can together be configured to cause the one or more processors to perform any or all parts of the method 200. For example, this may involve controlling the sensors 110 to perform the reference measurement and/or the source measurement. Further, it may involve using the source data to determine an estimate for the magnetic brain activity. The apparatus 100 may comprise a user interface for inputting control commands to the apparatus 100 and/or communicating the source data and/or information indicative thereof to a user. The user interface 330 may be arranged to prompt a user to initiate the reference measurement and/or the source measurement. The apparatus 100 may also be arranged to automatically obtain reference data and/or calculate a reference basis, for example daily, weekly or monthly. The apparatus 100 may be arranged to automatically perform the reference measurement in accordance with a schedule, which schedule may be adjustable and/or self-adjusting. The apparatus 100 may comprise one or more detectors 340 arranged to detect whether the reference measurement and/or the source measurement can be performed, for example by detecting whether any potential sources are present in the first volume or near the apparatus 100. The one or more detectors 340 may comprise, for example, a movement detector and/or a thermal detector, which may be arranged to detect an indication of the presence of a human in the first volume or near the apparatus 100. Prior to performing the reference measurement, the apparatus 100 may thus be arranged to use the one or more detectors 340 to evaluate whether the reference measurement can be performed. The apparatus 100 may also comprise one or more detectors for detecting an indication on whether a source is present at the apparatus for the source measurement. The apparatus 100 may comprise a separate measurement device 300 arranged to be coupled to the sensors 110. The measurement device 300 may comprise any combination of a processor 310, a memory 320, a user interface 330 and a detector 340.

The apparatus 100 may also comprise one or more detectors 340 arranged to measure the interference contribution during the source measurement. This may comprise one or more magnetometers and/or gradiometers. The measurement results obtained by these detectors may be used to improve the estimate for the magnetic brain activity of the source. These detectors 340 can be arranged outside the first volume and even outside the second volume to ascertain that they predominately measure the interference contribution, e.g. the magnetic fields from external sources. They may also be oriented away from the first volume.

As an example, the method has been compared to the traditional methods utilizing the SSS method. For this purpose a shielding factor is defined as the ratio of signal channel signal-vector norms before and after signal processing. The channels are picked to Ncomponent signal vector b (components b_{1 . . . N}) and the norm M is computed as

$$M ( t ) = \sqrt{{\sum }_{k = 1}^{N} {[ {b}_{k} ( t ) ]}^{2}} ,$$

where the sum runs over the length of the signal vector. Possible time dependence in any variable is indicated in parentheses with “(t)”. The norm is therefore the square root of the sum of squares of all the components of the signal vector. The shielding factor SF is the ratio of the norm from original data (M_{raw}) and processed data (Mₚₒₛₜ), SF=M_{raw}/Mₚₒₛₜ.

Shielding factor SF is estimated as a function of time. In the example, the mean values are tabulated over two-minute measurement duration. Channels with spurious artifacts have been excluded. SF has been evaluated separately for magnetometer and gradiometer channels for empty room recordings performed for nine TRIUX systems. For each system, recording has been analysed with large interference, EAS and IAS were not applied.

The shielding factors between different configurations and methods have been compared for nine systems and two recordings using two approaches for interference suppression:
1. SSS with fine-calibration. The fine-calibration adjustment for 306 channels was computed from large interference data.
2. Current method. The method as disclosed in the present application.

The shielding factors are collected in Table 1, where the type of magnetic sensors in the system is indicated on the first row and the channel geometry on the second row. For SSS, two fine-calibration models have been used: standard 1D-imbalance model and an improved 3D-imbalance model.

Both the current method and the SSS method with 3D-imbalance model have been found to outperform the standard SSS fine-calibration with 1D-imbalance model. The current method yields the best gradiometer shielding factor, where the improved result may be obtamed even with a reduced number of signal channels. Similar comparisons have been made also with the current method and the SSP method yielding similar results. With an extended set of tests, the current method has been found to provide an alternative to not only conventional SSS-based methods but conventional SSP-based methods as well. In particular, the current method may be used to improve shielding factors even without a fine calibration. It may also be used to significantly reduce the number of required channels for a MEG recording.

TABLE 1
Raw signal shielding factors SF for SSS with the 1D-
or 3D-imbalance model and for the current method.

| | magnetometers | | | gradiometers | | |
|---|---|---|---|---|---|---|
| | 306 | 306 | 306 | 306 | 306 | 204 |
| system | SSS 1D | SSS 3D | now | SSS 1D | SSS 3D | now |
| 3131 | 168 | 388 | 304 | 4 | 8 | 10 |
| 3132 | 234 | 538 | 510 | 4 | 10 | 31 |
| 3133 | 173 | 434 | 368 | 5 | 9 | 13 |
| 3134 | 379 | 775 | 747 | 5 | 10 | 44 |
| 3136 | 108 | 217 | 251 | 3 | 7 | 14 |
| 3137 | 339 | 742 | 882 | 6 | 15 | 72 |
| 3138 | 231 | 500 | 629 | 6 | 12 | 36 |
| 3140 | 303 | 768 | 705 | 5 | 12 | 38 |
| 3141 | 485 | 987 | 981 | 10 | 17 | 60 |
| mean | 269 | 594 | 597 | 5 | 11 | 35 |

The apparatus as described above may be implemented in software, hardware, application logic or a combination of software, hardware and application logic. The application logic, software or instruction set may be maintained on any one of various conventional computer-readable media. A “computer-readable medium” may be any media or means that can contain, store, communicate, propagate or transport the instructions for use by or in connection with an instruction execution system, apparatus, or device, such as a computer. A computer-readable medium may comprise a computer-readable storage medium that may be any media or means that can contain or store the instructions for use by or in connection with an instruction execution system, apparatus, or device, such as a computer. The examples can store information relating to various processes described herein. This information can be stored in one or more memories, such as a hard disk, optical disk, magneto-optical disk, RAM, and the like. One or more databases can store the information used to implement the embodiments. The databases can be organized using data structures (e.g., records, tables, arrays, fields, graphs, trees, lists, and the like) included in one or more memories or storage devices listed herein. The databases may be located on one or more devices comprising local and/or remote devices such as servers. The processes described with respect to the embodiments can include appropriate data structures for storing data collected and/or generated by the processes of the devices and subsystems of the embodiments in one or more databases.

All or a portion of the embodiments can be implemented using one or more general purpose processors, microprocessors, digital signal processors, micro-controllers, and the like, programmed according to the teachings of the embodiments, as will be appreciated by those skilled in the computer and/or software art(s). Appropriate software can be readily prepared by programmers of ordinary skill based on the teachings of the embodiments, as will be appreciated by those skilled in the software art. In addition, the embodiments can be implemented by the preparation of application-specific integrated circuits or by interconnecting an appropriate network of conventional component circuits, as will be appreciated by those skilled in the electrical art(s). Thus, the embodiments are not limited to any specific combination of hardware and/or software.

The different functions discussed herein may be performed in a different order and/or concurrently with each other.

Any range or device value given herein may be extended or altered without losing the effect sought, unless indicated otherwise. Also any example may be combined with another example unless explicitly disallowed.

Although the subject matter has been described in language specific to structural features and/or acts, it is to be understood that the subject matter defined in the appended claims is not necessarily limited to the specific features or acts described above. Rather, the specific features and acts described above are disclosed as examples of implementing the claims and other equivalent features and acts are intended to be within the scope of the claims.

It will be understood that the benefits and advantages described above may relate to one embodiment or may relate to several embodiments. The embodiments are not limited to those that solve any or all of the stated problems or those that have any or all of the stated benefits and advantages. It will further be understood that reference to ‘an’ item may refer to one or more of those items.

The term ‘comprising’ is used herein to mean including the method, blocks or elements identified, but that such blocks or elements do not comprise an exclusive list and a method or apparatus may contain additional blocks or elements.

Although the invention has been the described in conjunction with a certain type of apparatus and/or method, it should be understood that the invention is not limited to any certain type of apparatus and/or method. While the present inventions have been described in connection with a number of examples, embodiments and implementations, the present inventions are not so limited, but rather cover various modifications, and equivalent arrangements, which fall within the purview of prospective claims. Although various examples have been described above with a certain degree of particularity, or with reference to one or more individual embodiments, those skilled in the art could make numerous alterations to the disclosed examples without departing from the scope of this specification.

1. A magnetoencephalography apparatus comprising:
a plurality of magnetic sensors arranged for measurement of magnetic brain activity originating within a first volume, the plurality of magnetic sensors being arranged for positioning within a second volume, which is outside the first volume;
one or more processors coupled to the plurality of magnetic sensors for controlling the measurement of magnetic brain activity; and
one or more memories comprising computer program code, the one or more memories and the computer program code configured to cause the one or more processors to:
obtain a reference data corresponding to one or more measurements of the plurality of magnetic sensors in the absence of sources of magnetic brain activity in the first volume;
calculate from the reference data a reference basis, which represents magnetic activity in the absence of sources of magnetic brain activity in the first volume, in a signal space defined by the plurality of magnetic sensors;
obtain a source basis, which represents magnetic brain activity of a human brain positioned in the first volume, in the signal space defined by the plurality of magnetic sensors;
obtain a source data corresponding to one or more measurements of the plurality of magnetic sensors in the presence of a source of magnetic brain activity in the first volume;
add together the source basis and the reference basis to form a joint basis in the signal space defined by the plurality of magnetic sensors; and
determine an estimate for the magnetic brain activity of the source by parametrizing the source data in the joint basis.

2. The apparatus according to claim 1, wherein the magnetic sensors of the plurality of magnetic sensors are either all magnetometers or all gradiometers.

3. The apparatus according to claim 1, wherein the magnetic sensors of the plurality of magnetic sensors are gradiometers.

4. The apparatus according to claim 1, wherein the magnetic sensors of the plurality of magnetic sensors are planar gradiometers.

5. The apparatus according to claim 1, wherein the plurality of magnetic sensors is arranged to measure the magnetic brain activity with 48-256 signal channels.

6. The apparatus according to claim 1, wherein arranged to automatically perform the one or more measurements of the plurality of magnetic sensors to obtain the reference data.

7. The apparatus according to claim 1, wherein the source basis is obtained based on a deterministic solution for the Maxwell's equations for the magnetic brain activity of a human brain.

8. The apparatus according to claim 1, wherein the source basis is obtained based on a source model for the magnetic brain activity of a human brain comprising one or more stochastically positioned sources of magnetic brain activity.

9. A method comprising:
obtaining a reference data corresponding to one or more measurements of a plurality of magnetic sensors in the absence of sources of magnetic brain activity in a first volume; wherein the plurality of magnetic sensors have been arranged for measurement of magnetic brain activity originating within the first volume and positioned within a second volume, which is outside the first volume;
calculating from the reference data a reference basis, which represents magnetic activity in the absence of sources of magnetic brain activity in the first volume, in a signal space defined by the plurality of magnetic sensors;
obtaining a source basis, which represents magnetic brain activity of a human brain positioned in the first volume, in the signal space defined by the plurality of magnetic sensors;
obtaining a source data corresponding to one or more measurements of the plurality of magnetic sensors in the presence of a source of magnetic brain activity in the first volume;
add together the source basis and the reference basis to form a joint basis in the signal space defined by the plurality of magnetic sensors; and
determining an estimate for the magnetic brain activity of the source by parametrizing the source data in the joint basis.

10. The method according to claim 9, wherein the magnetic sensors of the plurality of magnetic sensors are either all magnetometers or all gradiometers, optionally planar gradiometers.

11. The method according to claim 9, wherein the plurality of magnetic sensors is arranged to measure the magnetic brain activity with 48-256 signal channels.

12. The method according to claim 9, wherein the one or more measurements of the plurality of magnetic sensors to obtain the reference data are performed automatically.

13. The method according to claim 9, wherein the source basis is obtained based on a deterministic solution for the Maxwell's equations for the magnetic brain activity of a human brain.

14. The method according to claim 9, wherein the source basis is obtained based on a source model for the magnetic brain activity of a human brain comprising one or more stochastically positioned sources of magnetic brain activity.

15. A computer program product comprising instructions which, when the program is executed by a computer, cause the computer to perform operation comprising:
obtaining a reference data corresponding to one or more measurements of a plurality of magnetic sensors in the absence of sources of magnetic brain activity in a first volume, wherein the plurality of magnetic sensors have been arranged for measurement of magnetic brain activity originating within the first volume and positioned within a second volume, which is outside the first volume;
calculating from the reference data a reference basis, which represents magnetic activity in the absence of sources of magnetic brain activity in the first volume, in a signal space defined by the plurality of magnetic sensors;
obtaining a source basis, which represents magnetic brain activity of a human brain positioned in the first volume, in the signal space defined by the plurality of magnetic sensors;
obtaining a source data corresponding to one or more measurements of the plurality of magnetic sensors in the presence of a source of magnetic brain activity in the first volume;
add together the source basis and the reference basis to form a joint basis in the signal space defined by the plurality of magnetic sensors; and
determining an estimate for the magnetic brain activity of the source by parametrizing the source data in the joint basis.

16. The computer program product according to claim 15, wherein the magnetic sensors of the plurality of magnetic sensors are either all magnetometers or all gradiometers, optionally planar gradiometers.

17. The computer program product according to claim 15, wherein the plurality of magnetic sensors is arranged to measure the magnetic brain activity with 48-256 signal channels.

18. The computer program product according to claim 15, wherein the one or more measurements of the plurality of magnetic sensors to obtain the reference data are performed automatically.

19. The computer program product according to claim 15, wherein the source basis is obtained based on a deterministic solution for the Maxwell's equations for the magnetic brain activity of a human brain.

20. The computer program product according to claim 15, wherein the source basis is obtained based on a source model for the magnetic brain activity of a human brain comprising one or more stochastically positioned sources of magnetic brain activity.
